(19) United States
(12) Patent Application Publication
(10) Pub. No.: US 2023/0124515 A1
(43) Pub. Date: Apr. 20, 2023

(54) NANOZYME LINKED BIOASSAY AND ASSOCIATED METHODS

(71) Applicant: Washington State University, Pullman, WA (US)

(72) Inventors: Dan Du, Pullman, WA (US); Yuehe Lin, Pullman, WA (US); Zhaoyuan Lyu, San Diago, CA (US); Shichao Ding, San Diago, CA (US)

(21) Appl. No.: 17/963,376

(22) Filed: Oct. 11, 2022

Related U.S. Application Data
(60) Provisional application No. 63/255,836, filed on Oct. 14, 2021.

(51) Int. Cl. B81C 1/00; C12Q 1/6816; G01N 21/78

(52) U.S. Cl. CPC B81C 1/00031; C12Q 1/6816; G01N 21/78; B82Y 5/00

(57) ABSTRACT

Single atom nanozymes and associated immunoassays, method of making, and method of using such immunoassays are described herein. For example, a method of making a single atom nanozyme includes forming a soft template having multiple nanoscale structures in an aqueous solution and adding a monomer and a metal containing salt into the aqueous solution. The metal containing salt causes polymerization of the monomer to form multiple nanostructures according to the nanoscale structures of the soft template. The method also includes coating the individual formed nanostructures with a confinement layer in the aqueous solution before pyrolyzing. During pyrolysis, the confinement layer at least restricts or completely prevents migration of atoms on the external surface of the individual nanostructures.

### CROSS REFERENCE TO RELATED APPLICATION(S

This application is a non-provisional of and claims priority to U.S. Provisional Application No. 63/255,836, filed on Oct. 14, 2021.

### BACKGROUND

Bioassay is a biochemical test that uses antibodies or antigens to measure a presence/concentration of target molecules. Detection targets of bioassay are often referred to as “analytes.” Example analytes include proteins, bacteria, viruses, or other macromolecules in serum, plasma, or urine. An antibody is a Y-shaped protein that can bind with unique molecular structures called “antigens.” Each tip at the “Y” shape of an antibody contains a paratope (analogous to a lock) that specifically corresponds to an epitope (analogous to a key) of an antigen. Thus, using an antibody with a distinct paratope one can precisely bind with an antigen having a corresponding epitope, and thus identify the antigen and/or a macromolecule that contains the antigen. In other applications, an antigen can also be used to bind an antibody with a distinct paratope in order to identify the antibody.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In bioassays, antibodies can be chemically linked with markers commonly referred to as “labels” for detection and measurement of antibodies bound to corresponding antigens. Example labels include enzymes, radioactive isotopes, and fluorogenic reporters that can produce a color change in a solution, emit radiation, fluoresce under light, be induced to emit light, or generate other detectable effects. For instance, in enzyme linked immunosorbent assays (ELISA), antibodies are chemically linked (e.g., covalently) with an enzyme, such as horseradish peroxidase (HRP). After binding antibodies with antigens in the analyte and subsequent washout, a “substrate” such as a combination of 3,3′,5,5′-tetramethylbenzidine (TMB) and hydrogen peroxide (H₂O₂) can be added to the solution. Upon contact with the substrate, the HRP linked to the antibodies can catalyze an oxidation reaction between the TMB and hydrogen peroxide (H₂O₂) to produce a color change. A spectrometry analysis on the color change can readily reveal the presence/concentration of the analyte in the sample as correlated to that of the HRP chemically linked to the antibodies.

ELISA technique utilizing HRP, however, may not reliably detect biomarkers at low concentrations that are characteristic of early stages of diseases. For example, during early stages of Alzheimer’s disease, the protein amyloid beta 1-40 (Aβ 1-40) can form insoluble toxic Aβ 1-40 aggregation, which can be used as a neuropathological biomarker to identify Alzheimer’s disease. The clinically relevant range of Aβ 1-40 is several tens to several hundred picograms per milliliter for patients presenting symptoms of Alzheimer’s disease. However, Alzheimer’s disease can begin in a human body years before any symptoms. As such, detecting Aβ 1-40 at low concentrations, such as sub-ten picogram per milliliter, would be helpful to estimate the risk or show the presence of Alzheimer’s disease at early stage. In addition, environmental factors such as pH and temperatures can significantly impact the shelf life of ELISA kits utilizing HRP. For instance, ELISA kits containing HRP typically are stored at low temperatures (e.g., 4° C.). Even at such conditions, the ELISA kits containing HRP can be stable for only a short period typically less than one year.

Several embodiments of the disclosed technology provide single-atom nano-enzymes or nanozymes (SANs) that can be chemically linked to antibodies in immunoassay to detect low concentration biomarkers and can be stable under harsh environmental conditions. A SAN can include a nanoscale structure in which at least some or all the catalytic active sites contain a metal moiety (e.g., iron) present as isolated single atoms stabilized by the support of or by bonding with additional atoms, such as nitrogen (N), carbon (C), or another metal. As described in more detail below, certain embodiments of the SANs can possess significantly increased catalytic capabilities while can remain stable for longer periods under harsh environmental conditions when compared to natural enzymes such as HRP.

In certain implementations, the SANs can include nanotubes individually having multiple surface single-atom active sites containing iron (Fe) atoms that can catalyze an oxidation reaction with hydrogen peroxide (H₂O₂). In one example, the SANs can include nanotubes of polypyrrole (H(C₄H₂NH)ₙH) having large numbers of iron, nitrogen, and carbon atoms. After pyrolysis, Fe—N—C active sites can be formed on the surfaces of the nanotubes. Iron (Fe) atoms incorporated into the Fe—N—C active sites can be at a concentration of about 0.40 atom% to about 1.0 atom% or greater on the external surface of the nanotubes. Molecular structure of an active site can include a central iron (Fe) atom covalently connected to four adjacent nitrogen (N) atoms, which in turn are individually and covalently connected to corresponding carbon (C) atoms in the nanotube structure. In additional examples, the active sites can also be based on other metals (e.g., zinc or cobalt) or formed on nanotubes, nanosprings, nanocoils, nanodots, or other suitable nanostructures formed from aniline, dopamine, carbon, or other suitable precursor materials.

Several embodiments of the disclosed technology also provide suitable methods of synthesizing the SANs with such single atom active sites as described above. In certain implementations, a method includes initially forming a soft template of nanoscale structures in an aqueous solution or deionized water. For example, methyl orange (MO) can be added to deionized water to form multiple micelles due to surface interaction of MO with water molecules in the aqueous solution or deionized water. Subsequently, a solution of pyrrole monomer and a solution of iron chloride (FeCl₃) can be added to the solution. The iron chloride (FeCl₃) not only provides the iron (Fe) atoms for forming the Fe—N—C active sites but can also act as an oxidizing agent to facilitate the polymerization of the pyrrole monomers. As such, nanotubes of polypyrrole can be formed based on the soft template of MO. The formed nanotubes can then undergo a pyrolysis operation to derive the target SANs with surface Fe—N—C active sites.

In accordance with additional embodiments of the disclosed technology, the method can also include forming a confinement layer on the polypyrrole nanotubes prior to pyrolysis of the nanotubes. A confinement layer can include a solid molecular structure that covers at least a portion of or the entire surface of the individual nanotubes. For example, potassium permanganate (KMnO₄) can be added to be reduced and form a magnesium oxide (MnO₂) coating on the external surfaces of the individual polypyrrole nanotubes. In other examples, the confinement layer can also include a layer of silicon oxide (SiO₂), titanium oxide (TiO₂), a polymer, an ionic liquid, or other suitable materials formed via an oxidation, reduction, or other suitable chemical/physical transformations.

After forming the confinement layer (e.g., the magnesium oxide coating) on the nanotubes, the method can include performing one or more pyrolysis operations to derive the target SANs. For instance, in one implementation, a first pyrolysis operation in a nitrogen (N₂) environment can be initially performed. Then, the method can include removing any aggregated iron (Fe) atoms and the confinement layer (e.g., magnesium oxide (MnO₂) coating) on the nanotubes via, for instance, acid leaching using sulfuric acid (H₂SO₄). Then, the method includes another heat treatment operation in an ammonia (NH₃) environment to obtain the SANs. In other embodiments, the method can include a single pyrolysis operation and/or other suitable operations prior, during, or after pyrolyzing the nanotubes.

It is believed that the confinement layer, such as the magnesium oxide (MnO₂) coating can confine atoms (e.g., the iron atoms) on the surfaces of the nanotubes to reduce migration and aggregation of various precursors such as iron (Fe) during pyrolysis. It is believed that surface and/or internal atoms of the nanotubes tend to migrate under the high thermal energy environment during pyrolysis. As such, atoms, such as iron (Fe) atoms that form the individual single-atom active sites can aggregate with additional iron (Fe) atoms to form aggregated iron (Fe). The iron aggregation in turn reduces the number of iron (Fe) atoms available to form the single-atom active sites on the nanotubes. Thus, forming a solid barrier with the confinement layer can reduce, obstruct, or disrupt such migration such that a high atomic distribution of single atom iron (Fe) can be achieved. As such, high numbers of active sites can be produced on the surfaces of the nanotubes.

The obtained SANs from the foregoing process can then be chemically linked to suitable antibodies in immunoassays as labels. For example, in one embodiment, the foregoing obtained SANs can be treated with a solution of N-(3-dimethylamino propyl)-N′-ethylcarbodiimide hydrochloride (EDC) and N-hydroxysuccinimide (NHS), and then modified with streptavidin (SA) to bind biotinylated Aβ 1-40. The biotin can react with SA-conjugated labels, forming the strongest known non-covalent bond between a protein and a ligand. Notably, the interaction is rapid and maintains robustly in extreme conditions of pH and temperature levels.

As such, in the above example, the obtained streptavidin-modified SANs can be used to substitute HRP-streptavidin to enhance the detection performance of Aβ 1-40 and other biomarkers. As described in more detail herein, the limit of detection (LOD) using the streptavidin-modified SANs can be an order of magnitude lower than the LOD of traditional ELISA technique based on HRP. In other embodiments, the obtained SANs can be chemically linked to other suitable antibodies in immunoassays for detecting other proteins, bacteria, viruses, or other suitable detection targets.

### BRIEF DESCRIPTION OF THE DRAWINGS

FIG. 1 is a schematic diagram of an example single atom nanozyme linked immunosorbent assay in accordance with embodiments of the disclosed technology.

FIGS. 2A-2F are schematic and flow diagrams showing chemical structures of a process suitable for synthesizing SANs of FIG. 1 in accordance with embodiments of the disclosed technology.

FIG. 3A is a transmission electron microscopy (TEM) image of example SANs formed using the process of FIGS. 2A-2F in accordance with embodiments of the disclosed technology.

FIG. 3B is a high-resolution transmission electron microscopy (HRTEM) image of example SANs formed using the process of FIGS. 2A-2C in accordance with embodiments of the disclosed technology.

FIG. 3C shows an example result of energy-dispersive X-ray spectroscopy (EDS) performed on SANs formed using the process of FIGS. 2A-2C in accordance with embodiments of the disclosed technology.

FIG. 3D is a TEM image of Fe-Nₓ SANs and EDS elemental mapping results of C, N, and Fe of SANs formed using the process of FIGS. 2A-2C in accordance with embodiments of the disclosed technology.

FIG. 3E is an example Fourier transform infrared spectra of the Fe-Nx SANs and SA-labeled Fe-Nx SANs in accordance with embodiments of the disclosed technology. The strong peak at 1638 cm⁻¹ which corresponds to the amide I show that streptavidin is already successful labeled on Fe-Nx SANs.

FIGS. 3F and 3G are example TEM images showing Morphology of PPy nanotubes and MnO₂ coated PPy nanotubes in accordance with embodiments of the disclosed technology.

FIG. 3H is an example nitrogen (N2) adsorption-desorption isotherm of Fe-NxSANS in accordance with embodiments of the disclosed technology.

FIG. 3I is an example photoelectron spectroscopy (XPS) spectrum of Fe-Nx SANs in accordance with embodiments of the disclosed technology.

FIG. 3J is a schematic diagram illustrating a structure of natural HRP; and FIG. 3K is a schematic diagram illustrating a structure of iron (II) phthalocyanine (FePc) in accordance with embodiments of the disclosed technology.

FIGS. 4A and 4B show example high-resolution nitrogen 1s and iron 2p spectra of Fe-Nx SANs, respectively, formed using the process of FIGS. 2A-2C in accordance with embodiments of the disclosed technology.

FIG. 4C shows example nitrogen, oxygen, and iron contents in Fe-Nx SANs formed using the process of FIGS. 2A-2C in accordance with embodiments of the disclosed technology.

FIG. 4D shows example iron K-edge X-ray absorption near-edge structure (XANES) spectra of Fe-Nx SANs formed using the process of FIGS. 2A-2C in accordance with embodiments of the disclosed technology and reference samples of FePc, Fe foil, FeO, and Fe₂O₃.

FIG. 4E shows an example Fourier-transform extended X-ray absorption fine structure (EXAFS) curve of Fe-Nx SANs, FePc, and Fe foil in accordance with embodiments of the disclosed technology.

FIG. 4F shows an example scanning TEM image of Fe-Nx SAN formed using the process of FIGS. 2A-2C in accordance with embodiments of the disclosed technology.

FIG. 5A is a schematic free energy diagram illustrating a catalytic operation of Fe-Nx SANs formed using the process of FIGS. 2A-2C in accordance with embodiments of the disclosed technology.

FIG. 5B is an example absorbance-time curve of TMB chromogenic reactioncatalyzed by Fe-Nx SANs and the corresponding magnified initial linear portion in accordance with embodiments of the disclosed technology.

FIG. 5C is an example specific activity plot of Fe-Nx SANs formed using the process or FIGS. 2A-2C in accordance with embodiments of the disclosed technology.

FIGS. 6A and 6B are example steady-state kinetics curves of Fe-Nx SANs toward TMB and H₂O₂, respectively, in accordance with embodiments of the disclosed technology.

FIGS. 6C and 6D are example relative activity plots of Fe-Nx SANs with respect to temperature and pH, respectively, in accordance with embodiments of the disclosed technology.

FIG. 7A is a schematic illustration of SANs-linked immunosorbent assay (SANs-LISA) for the detection of Aβ 1-40 in accordance with embodiments of the disclosed technology.

FIG. 7B is an example curve of SAN-LISA for the detection of Aβ 1-40 ranging from 1 picogram/milliliter to 2000 picogram/milliliter in accordance with embodiments of the disclosed technology.

FIG. 7C illustrates example absorbance spectra of various concentrations of Aβ 1-40 detected by SAN-LISA in accordance with embodiments of the disclosed technology.

FIG. 7D shows example standard curves of SAN-LISA (Aβ 1-40 ranging from 1 to 2000 picogram/milliliter) and ELISA (Aβ 1-40 ranging from 100 picogram/milliliter to 100 picogram/milliliter) in accordance with embodiments of the disclosed technology.

FIG. 7E shows example specificity plot of SAN-LISA (Aβ 1-40 of 400 picogram/milliliter; Carcinoembryonic antigen (CEA), Bovine serum albumin (BSA), and Immunoglobulin G (IgG) of 5 ng/mL, respectively, in accordance with embodiments of the disclosed technology.

### DETAILED DESCRIPTION

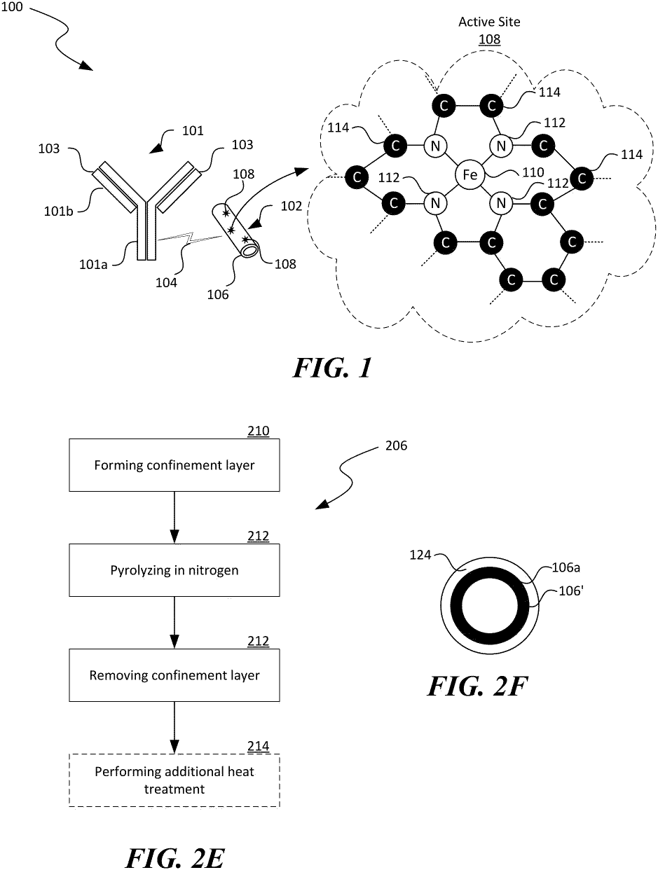

Certain embodiments of systems, devices, articles of manufacture, and processes for nanozyme linked bioassay and associated methods of manufacturing and using are described below. Though the disclosure below uses immunoassay as an example for the application of the nanozyme, in other implementations, the nanozyme can also be used with DNA or aptamer assays or other suitable types of assays. In the following description, specific details of components are included to provide a thorough understanding of certain embodiments of the disclosed technology. A person skilled in the relevant art will also understand that the disclosed technology may have additional embodiments or may be practiced without several of the details of the embodiments described below with reference to FIGS. 1A-7E.

As used herein, the term “immunoassay” generally refers to a biochemical test that uses antibodies or antigens to measure a presence/concentration of target molecules generally referred to as “analytes.” Example analytes include proteins, bacteria, viruses, or other macromolecules in serum, plasma, or urine. Immunoassays rely on ability of an antibody to recognize and bind specific molecular structures called “antigens”, or vice versa. An antibody is a Y-shaped protein that can bind with an antigen unique on macromolecules. Each tip of the “Y” of an antibody contains a paratope (analogous to a lock) that specifically corresponds to an epitope (analogous to a key) of an antigen. Thus, using an antibody with a distinct paratope one can precisely bind with an antigen having a corresponding epitope, and thus identify the macromolecule that contains the antigen. Conversely, an antigen can also be used to bind with and identify a specific antibody. In other words, the antibody is the analyte instead of the antigen.

Also used herein, a “nanozyme” generally refers to a nanoscale structure having catalytic capabilities to facilitate an oxidation, reduction, or other types of reactions for immunoassay. “Nanoscale structures” or “nanostructures” generally refers to structures having at least one dimension at nanoscale between microscopic and molecular scales. Example ranges of nanoscale can be from 0.1 nm to 100 nm or other suitable ranges. To illustrate, a nanostructure can be a nanotube that has a tubular shape with at least one of a thickness or diameter at nanoscale, e.g., between 0.1 nm and 100 nm. Such a nanotube, however, may have a length that exceeds the nanoscale. Other examples of nanostructures can include nanosprings, nanocoils, nanodots, or other structures with suitable shapes and dimensions in nanoscale.

As used herein, a “confinement layer” generally refers to a layer of material on an external surface of a structure (e.g., a nanoscale structure) that at least restricts or even prevents atoms of the structure from migrating during processing operations such as pyrolysis. A confinement layer can be formed in various ways. For example, as described in more detail herein, a confinement layer of magnesium oxide (MnO₂) can be formed by reducing potassium permanganate (KMnO₄). In other examples, a confinement layer of silicon oxide (SiO₂), titanium oxide (TiO₂), a polymer, an ionic liquid, or other suitable materials can be formed via chemical vapor deposition, atomic layer deposition, or other suitable techniques.

Further, as used herein, an “active site” generally refers to a portion of a nanostructure that can catalyze an oxidation or other types of reaction of hydrogen peroxide (H₂O₂) or other suitable compositions. For example, as described in more detail herein, an example active site can include a central iron (Fe) atom covalently connected to multiple nitrogen (N) atoms, which in turn are covalently connected to additional carbon (C) atoms. Such an example active site can catalyze an oxidation reaction of hydrogen peroxide (H₂O₂) by readily disassociating one hydroxyl group from a molecule of hydrogen peroxide (H₂O₂). In other examples, an active site can also contain zinc (Zn), cobalt (Co), or other suitable central metal atoms.

ELISA technique utilizes antibodies chemically linked with an enzyme such as HRP to detect target analytes. ELISA technique utilizing HRP, however, may not reliably detect biomarkers at low concentrations characteristic of early stages of diseases. For example, utilizing HRP, ELISA may not reliably detect the protein amyloid beta 1-40 (Aβ 1-40), which can be used as a neuropathological biomarker to identify Alzheimer’s disease. In addition, environmental factors such as pH, temperatures, and shelf life can significantly impact the effectiveness of ELISA kits utilizing HRP. For instance, kits containing HRP typically are stored at low temperatures (e.g., 4° C.). Even under such conditions, the kits containing HRP can only be stable for a short period typically less than one year.

Several embodiments of the disclosed technology provide single-atom nano-enzymes or nanozymes (SANs) that can be chemically linked to antibodies in an immunoassay to detect low concentration biomarkers. As used herein, a SAN generally refers to a nanoscale structure in which at least some or all the catalytic active sites contain a metal molecule (e.g., iron) existing as isolated single atoms stabilized by the support of or by bonding with additional atoms of the nanoscale structure, such as nitrogen (N), carbon (C), or another metal. As described in more detail below with reference to FIG. 1A, embodiments of the SANs can possess significantly increased catalytic capabilities while can remain stable for longer periods when compared to natural enzymes such as HRP.

FIG. 1 is a schematic diagram of an example single atom nanozyme (SAN)-linked immunosorbent assay (LISA) 100 in accordance with embodiments of the disclosed technology. As shown in FIG. 1, in the illustrated embodiment, the SAN-LISA 100 includes an antibody 101 chemically linked to a SAN 102 via a bond 104. The antibody 101 can include multiple protein chains 101a and 101b arranged in a “Y” shape. Each tip of the “Y” shape of an antibody contains a paratope 103 (analogous to a lock) that specifically corresponds to an epitope (analogous to a key) of an antigen (not shown). The bond 104 can be a covalent bond, hydrogen bond, or other suitable chemical/physical interactions that connect the SAN 102 to the antibody 101. In other embodiments, the SAN 102 can be chemically linked to the antibody 101 via intermediate structures (not shown) such as other proteins or other suitable materials.

As shown in FIG. 1, the SAN 102 can include a nanotube 106 having multiple active sites 108 (shown as stars for illustration purposes) configured to catalyze an oxidation, reduction, or other suitable reactions on the external surface of the nanotube 106. For instance, in one implementation, the active sites 108 can be configured to catalyze an oxidation reaction of hydrogen peroxide (H₂O₂) by disassociating a hydroxyl group from hydrogen peroxide (H₂O₂). Though the SAN 102 is shown in FIG. 1 as having the shape and dimension of a nanotube, in other embodiments, the SAN 102 can also have the shape and dimension of a nanospring, nanocoil, nanodot, or other suitable configurations. In other implementations, the active sites 108 can be configured to catalyze orfacilitate other suitable types of chemical reactions.

In certain embodiments, the individual active sites 108 can include a central metal atom covalently connected to additional atoms of the material forming the nanotube 106. For instance, in one example, the active site 108 includes an iron (Fe) atom 110 covalently connected to four nitrogen (N) atoms 112, which in turn are covalently connected to additional carbon (C) atoms 104 of polypyrrole (H(C₄H₂NH)ₙH) after pyrolysis to form an iron-nitrogen-carbon (Fe—N—C) active site 108. Iron (Fe) atoms 110 incorporated into the FeN—C active sites 108 can be at a concentration of about 0.40 atom% to about 1.0 atom% or greater on the external surface of the nanotube 106. In additional embodiments, the active sites 108 can also be based on other metals (e.g., zinc or cobalt) or formed on nanotubes, nanosprings, nanocoils, nanodots, nanosheets, or other suitable nanostructures formed from aniline, dopamine, carbon, or other suitable precursor materials. As described in more detail below, embodiments of the SAN 102 can possess significantly increased catalytic capabilities while can remain stable for longer periods when compared to natural enzymes such as HRP.

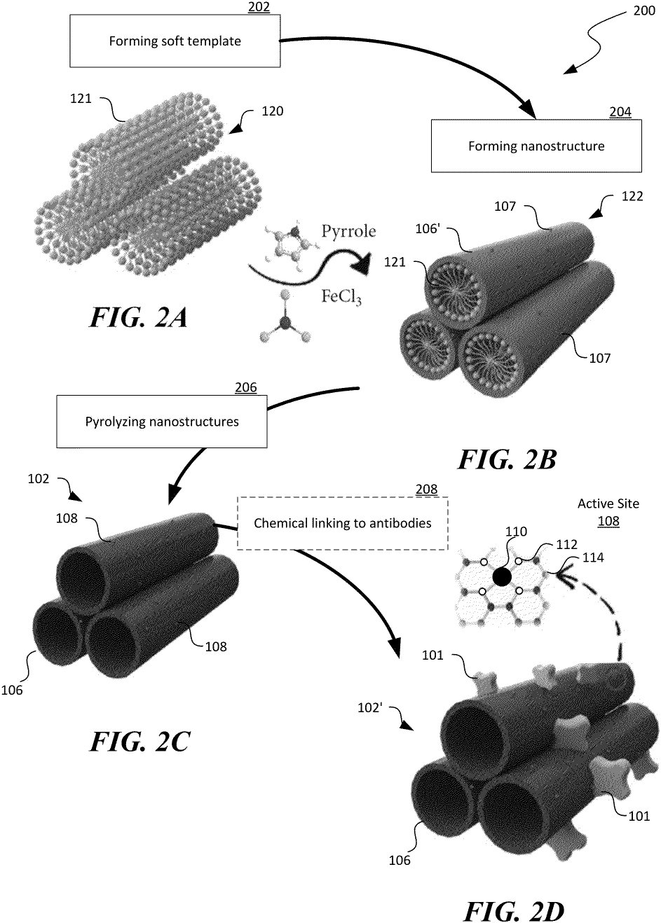

FIGS. 2A-2C are schematic and flow diagrams showing chemical structures of a process 200 suitable for synthesizing SANs 106 of FIG. 1 in accordance with embodiments of the disclosed technology. As shown in FIG. 2A, the process 200 can include initially forming a soft template 120 in an aqueous solution or deionized water at stage 202. For example, methyl orange (MO) can be added to deionized water to form multiple micelles 121 due to surface interaction of MO with water molecules (not shown) in the aqueous solution or deionized water.

Subsequently, the process 200 can include forming nanostructures based on the soft template 120 at stage 204. In the illustrated example in FIG. 2B, the nanostructures include nanotube precursors 106′. In other examples, the nanostructures can also include nanosprings, nanocoils, nanodots, nanosheets, or other suitable nanoscale structures. In one implementation, a solution of pyrrole monomer and a solution of iron chloride (FeCl₃) can be added to the solution. The iron chloride (FeCl₃) not only provides the iron (Fe) atoms 107 loaded in the nanotube precursors 106′ of the polypyrrole for forming the Fe—N—C active sites 108 (shown as small dots on the external surfaces of the nanotubes 106 in FIG. 2C) but can also act as an oxidizing agent to facilitate the polymerization of the pyrrole monomers. As such, nanotube precursors 106′ of polypyrrole can be formed based on the soft template 120 of MO. The formed nanotube precursors 106′ can then undergo a pyrolysis operation to derive the SANs 102 with the surface Fe—N—C active sites 108 at stage 206, as shown in FIG. 2C. As described in more detail below with reference to FIG. 2E, during pyrolysis, a confinement layer 124 (shown in FIG. 2E) over the external surfaces 106a of the nanotube precursors 106′ can be used to at least reduce or even prevent migration of atoms on the external surfaces 106a of the nanotube precursors 106′, and thus increasing an atomic density of the Fe—N—C active sites 108 on the formed nanotubes 106 as shown in FIG. 2C.

The obtained SANs 102 from the foregoing operation can then be chemically linked to suitable antibodies 101 in immunoassays as labels at stage 208. For example, in the illustrated embodiment, the obtained SANs 102 can be treated with a solution of N-(3-dimethylamino propyl)-N′-ethylcarbodiimide hydrochloride (EDC) and N-hydroxysuccinimide (NHS), and then modified with streptavidin (SA) to bind biotinylated Aβ 1-40 antibody. The biotin can react with SA-conjugated labels to form the strongest known non-covalent bond between a protein (e.g., SA) and a ligand. Notably, the interaction is rapid and maintains robustly in extreme conditions of pH and temperature levels.

As such, in the above example, the obtained streptavidin-modified SANs 102′ can be used to substitute HRP-streptavidin to enhance the detection performance of Aβ 1-40 and other biomarkers. As shown in FIG. 2D, the nanotubes 106 can individually include multiple active sites 108 each having an iron (Fe) atom 110 covalently connected to four nitrogen (N) atoms 112, which in turn are covalently connected to additional carbon (C) atoms 114 to form a Fe—N—C active site 108. Iron (Fe) atoms incorporated into the Fe—N—C active sites 108 can be at a concentration of about 0.40 atom% to about 1.0 atom% or greater on the external surface of the nanotube 106. As described in more detail herein, the limit of detection (LOD) using the streptavidin-modified SANs 102 can be an order of magnitude lower than the LOD of traditional ELISA technique based on HRP. In other embodiments, the obtained SANs 102 can be chemically linked to other suitable antibodies in immunoassays for detecting other proteins, bacteria, viruses, or other suitable detection targets.

As shown in FIG. 2E, in accordance with additional embodiments of the disclosed technology, pyrolyzing the nanostructures can also include forming a confinement layer 124 (shown in FIG. 2F) on the nanotubes 106 (FIG. 2B) prior to pyrolysis of the formed nanotubes 106 at stage 210. A confinement layer generally refers to a solid molecular structure that covers at least a portion of or the entire surface of the individual nanotubes 106. For example, in one implementation, potassium permanganate (KMnO₄) can be added to the aqueous solution to be reduced to form a magnesium oxide (MnO₂) coating on the external surfaces of the individual nanotubes. In other examples, the confinement layer can also include a layer of silicon oxide (SiO₂), titanium oxide (TiO₂), a polymer, an ionic liquid, or other suitable materials formed via an oxidation, reduction, or other suitable chemical/physical transformations. FIG. 2F is a cross-sectional diagram illustrating an example confinement layer 124 over an external surface 106a of the nanotube 106.

Referring back to FIG. 2E, after forming the confinement layer 124 (e.g., the magnesium oxide coating) on the nanotubes 106, the pyrolysis operation 206 can include performing one or more pyrolysis procedures to derive the SANs 106. For instance, in one implementation, a first pyrolysis operation in a nitrogen (N₂) environment can be initially performed at stage 212. Subsequently, the pyrolysis operation 206 can include performing removing the confinement layer 124 and any aggregated iron (Fe) atoms on the external surfaces of the nanotubes 106 via, for example, acid leaching. Then, the pyrolysis operation 206 can include performing a second heat treatment procedure in an ammonia (NH₃) environment to obtain the SANs 102 at stage 214. In other embodiments, the pyrolysis operation 206 can include performing a single pyrolysis procedure and/or other suitable operations prior, during, or after pyrolyzing the nanotubes 102.

It is believed that the confinement layer 124, such as the magnesium oxide (MnO₂) coating can confine atoms (e.g., the iron atoms) on the external surfaces 106a (shown in FIG. 2F) of the nanotubes 106 to reduce migration and aggregation of various precursors such as iron (Fe) atoms 110 (shown in FIG. 2D) during pyrolysis. It is believed that surface and/or internal atoms of the nanotubes 106 tend to migrate under the high thermal energy environment during the pyrolysis operation 206. Atoms, such as iron (Fe) that form the individual single-atom active sites can aggregate with additional iron (Fe) atoms to form aggregated iron (Fe). The iron aggregation in turn reduces the number of iron (Fe) atoms available to form the single-atom active sites 108 on the nanotubes 106. Thus, forming a solid barrier with the confinement layer 124 can reduce, obstruct, or disrupt such migration such that a high atomic distribution of single atom iron (Fe) atoms 110 can be achieved. As such, high numbers of active sites 108 can be produced on the external surfaces 106a of the nanotubes 106.

### Experiments

Certain experiments were conducted according to embodiments of the process 200 shown in FIGS. 2A-2C to synthesize SANs 102. The procedures and results are discussed in more detail below.

### Instruments and Characterization

Images of materials were obtained by TEM (Tecnai G2 T20, 200 kV; JEOM Grand ARM300F, 300 kV); elemental analysis was conducted by X-ray photoelectron spectroscopy (XPS, Escalab 250, Al Kα). The X-ray absorption spectroscopy measurement at Fe K-edge was performed at the Advanced Photon Source (APS) on the bending-magnet beamline 9-BM-B with electron energy cof 7 GeV and average current of 100 mA. The radiation was monochromatized by a Si (111) double-crystal monochromator. All absorption spectra and fluorescence spectra were performed by Tecan Safire2 Multi-Mode Microplate Reader. The specific surface area of the sample was investigated with an automatic volumetric sorption analyzer (ASAP 20209 M) which N₂ acts as the adsorbate at -196° C.

### Evaluation of the Peroxidase-like Properties of Fe—Nx SANs

Peroxidase-like properties of Fe—Nx SANs were studied following the protocol (Nature protocols, 2018, 13(7): 1506). Specifically, TMB was used as a substrate to verify the peroxidase-like feature of Fe—Nx SANs. In a typical measurement, Fe—Nx SANs was dispersed in HAc—NaAc buffer with PH = 3.6 and distributed into a 96-well plate. Then 100 µL TMB (10 mg/mL in DMSO) was added. The mixture was incubated under 37° C. in dark for 1 minute, then H₂O₂ was added to final concertation of 1 M. The reaction-time curve of Fe-Nx SANs was plotted using the absorbance at 652 nm against the reaction time. The catalytic activity units (U) was evaluated by detecting the absorbance at 652 nm immediately and recorded at a 10 s interval within 700 s. After subtracting the background, the nanozyme activity expressed in units (U) was calculated according to the following equation:

$${b}_{\text{nanozyme}} = \frac{V}{ε l} \times \frac{Δ A}{Δ t}$$

In which b_{nanozyme} refers to the nanozyme activity (U), V is volume of the reaction solution (µL), ε is the molar absorption coefficient of TMB substrate (39,000 M⁻¹ cm⁻¹ at 652 nm), I is the optical path length through reaction solution (cm) and ΔA/Δt is the initial rate (within 1 min) of the absorbance change (min⁻¹).

When using different amounts of Fe—Nx SANs to measure the peroxidase-like activity, the specific nanozyme activity was determined by the following equation:

$${a}_{\text{nanozyme}} = \frac{{b}_{\text{nanozyme}}}{m}$$

where a_{nanozyme} is the specific activity of nanozyme (U mg⁻¹) and m is the nanozyme amount (mg).

For the steady-state kinetic measurements of peroxidase-like Fe—Nx SANs, 10 µL TMB solution with different concentrations (from 0 to 3.5 mM) and a certain volume of 1 M H₂O₂ solution were added to NaAc—HAc buffer (pH 3.6) to a final concentration of 1 M. After 50 µL of 1 µg/mL Fe—Nx SANs solution was added and mixed for reaction. The absorbance at 652 nm was immediately recorded at a 10-second interval within 60 seconds. Then, the initial rates of the chromogenic reaction upon different TMB concentrations were obtained. The substrate concentration-dependent reaction rate curves were fitted with Michaelis-Menten model and Michaelis constant Kₘ and K_{cat} were calculated according the following Michaelis-Menten equation:

$$v = \frac{{v}_{max} \left[S\right]}{{K}_{m} + \left[S\right]} {K}_{c a t} = \frac{{υ}_{max}}{\left[E\right]}$$

where v is the initial rate of the chromogenic reaction, [S] is the TMB concentration and [E] is the nanozyme concentration (M). Finally, peroxidase-like activity of Fe—Nₓ SANs was analyzed and the steady-state kinetics properties of Fe—Nₓ SANs were also evaluated and compared with HRP.

### DFT Computational Details

Vienna ab initio simulation package (VASP) was conducted to all calculations with the with plane-wave pseudopotential method. Generalized gradient approximation (GGA) was utilized to describe the electronic exchange and related effects with Perdew-Burke-Ernzerhof (PBE) functions, while all-potential projector augmented wave (PAW) method was performed to describe the core electrons. Plane wave expansion utilized an energy cutoff of 600 eV, and the force on the relaxed atom was less than -0.03 eV/Å. The Van der Waals (VdW) corrections of DFT-D3 and spin-polarization were considered in all calculations. To study the structure and catalytic mechanism, Fe—N₄ sites embedded in a periodic (6 × 6) graphene supercell model were established, in which the vacuum region between the periodic plates was 20Å. The Brillouin zone was sampled using a (3 × 3 × 1) k-point grid generated by the Gamma scheme. The adsorption energy (E) was calculated according to

$$\text{E} \text{=} \text{EPGM-SACs} \text{-} \left({\text{E}}_{\text{SACs}} \text{+} {\text{E}}_{\text{PGM}}\right)$$

where E_{PGM}, E_{SACs}, and Eg_{as-SACs} represent the energies of the produced gas molecule, the clean Fe-doped carbon surface, and the corresponding adsorbed gas molecule on Fe-doped carbon surface, respectively.

### Preparation of Fe—Nx SANs

500 mg of MO was dissolved in deionized water. Then, 5 grams of iron chloride (FeCl₃) and 1.5 milliliter pyrrole were added to the solution under vigorous stirring to form Fe³⁺ doped polypyrrole (PPy) nanotubes. Magnesium oxide (MnO₂) coated PPy nanotubes were then prepared by dispersing a certain amount of potassium permanganate (KMnO₄) into the solution. The resulting product from the solution was then pyrolyzed at 900° C. under a nitrogen (N₂) atmosphere. The magnesium oxide (MnO₂) coating was then removed by acid leaching for 8 hours with 5% H₂SO₄ (v/v). Finally, the Fe—Nx SANs were obtained after a second heat treatment at 900° C. under ammonia (NH₃).

### Fabrication of SA Labeled Fe—Nx SANs

Initially, the synthesized Fe—Nx SANs were shattered under vigorous sonication and dispersed in phosphate buffered saline (PBS) (0.5 mg/ml), then adjusted by potassium carbonate (K₂CO₃) to reach a pH of about 6.0 and ultrasonicated for 1 hour. Then, the solution was activated by N-(3-dimethylaminopropyl)-N′-ethylcarbodiimide (EDC: 2 mg/mL) and N-hydroxysuccinimide (NHS: 4 mg/mL) under shaking for 30 minutes. Then, the solution was centrifuged and washed three times to form activated Fe—Nx SANs. SA (100 µg/ml in PBS) was incubated with activated Fe—Nx SANs at 37° C. for 1 hour. The mixture was then centrifuged for three times to remove unbonded SA. Then, the products were passivated with 1% bovine serum albumin (BSA) for 30 minutes and dispersed in 1 ml of PBS before the SA labeled Fe—Nx SANs were reduced to nanoscale sizes via an intense ultrasound treatment.

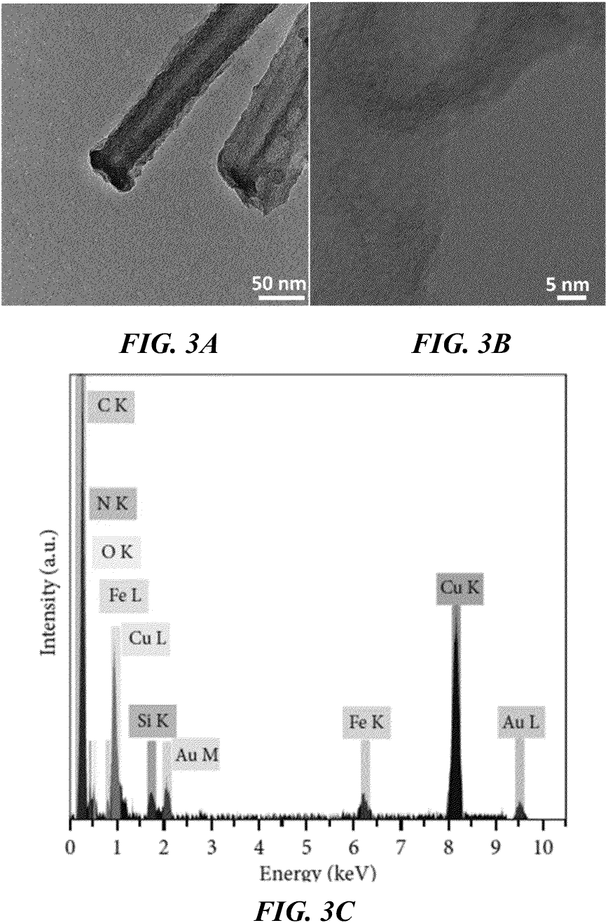

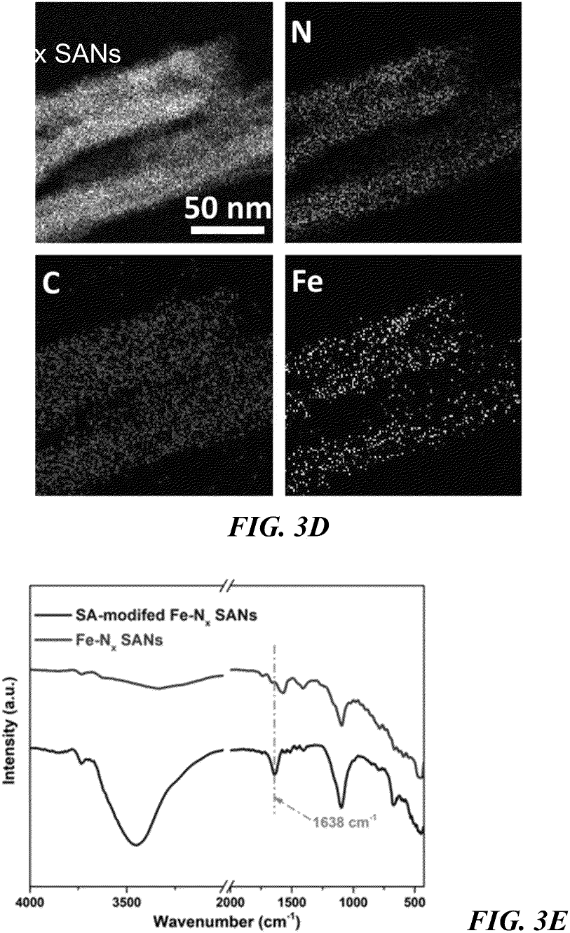

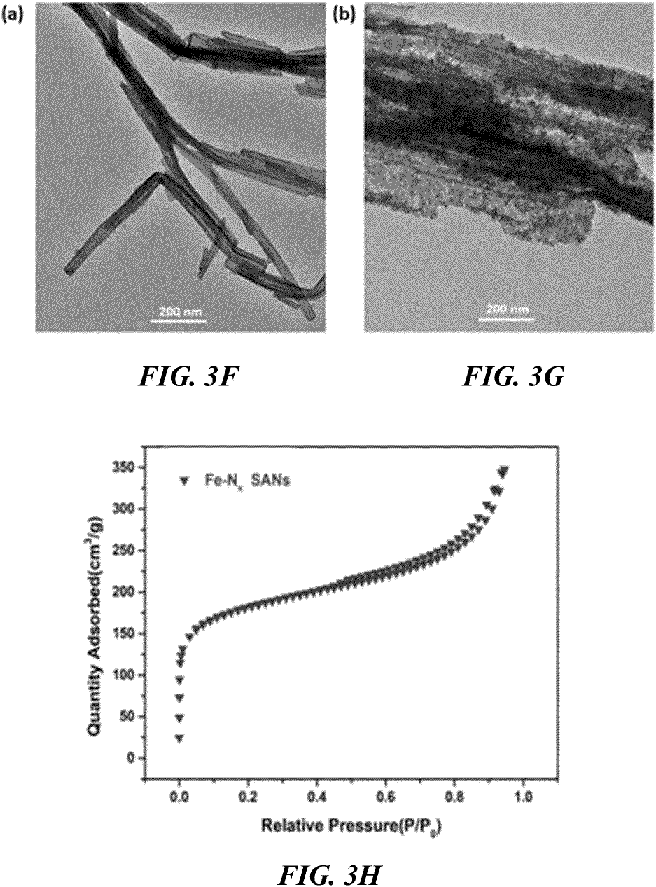

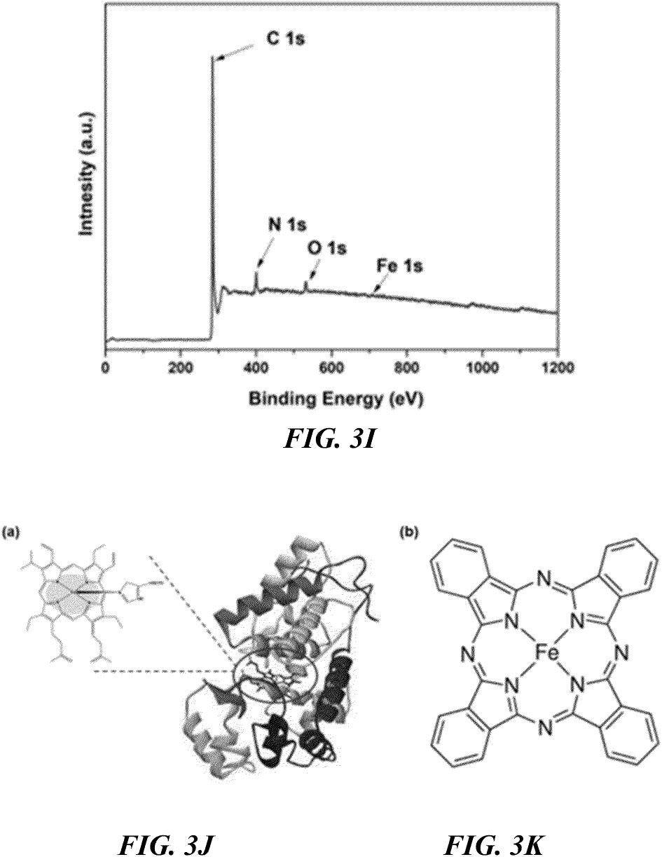

FIG. 3A is a transmission electron microscopy (TEM) image of example SANs formed using the process of FIGS. 2A-2C in accordance with embodiments of the disclosed technology. The morphologies of PPy nanotubes and MnO₂ coated PPy nanotubes were confirmed by transmission electron microscopy (TEM) as shown in FIG. 3E while the morphologies of PPy nanotubes and MnO₂ coating PPy nanotubes were confirmed by TEM as shown in FIGS. 3F and 3G. As shown in FIG. 3A, the Fe-Nₓ SANs had a well-defined nanotube structure with a diameter of around 50 nm. FIG. 3B is a high-resolution transmission electron microscopy (HRTEM) image of example SANs formed using the process of FIGS. 2A-2C in accordance with embodiments of the disclosed technology. As shown in FIG. 3B, distorted graphite layers were found in Fe—Nx SANs by high-resolution TEM (HRTEM). It is believed that this graphite structure can provide enriched defects and nanopores that can anchor atomic Fe—Nx moieties. N2 adsorption/desorption test was carried out to evaluate detailed textural structure, as shown in FIG. 3H.

FIG. 3C shows an example result of energy-dispersive X-ray spectroscopy (EDS) performed on SANs formed using the process of FIGS. 2A-2C in accordance with embodiments of the disclosed technology. As shown in FIG. 3C, EDS elemental analysis demonstrated that the Fe—Nx SANs were comprised of C, N and Fe. Here, the absence of the Mn signal indicated that the MnO₂ coating was removed successfully. The Si, Cu, and Au signals were introduced by TEM (such as EDS probes or TEM grid), which was shown in the X-ray photoelectron spectroscopy (XPS) spectrum in FIG. 3I. FIG. 3D is a TEM image Fe—Nx SANs and EDS elemental mapping results of C, N, and Fe of SANs formed using the process of FIGS. 2A-2C in accordance with embodiments of the disclosed technology. As shown in FIG. 3D, various elements were distributed generally uniformly in the Fe—Nx SANs, indicating that the Fe—Nx were incorporated into the PPy matrix. Also, no Fe clusters were observed. It is believed that the absence of Fe clusters was due to the aggregated Fe species being removed during acid treatment, and thus the remaining Fe atoms existed as isolated single atoms.

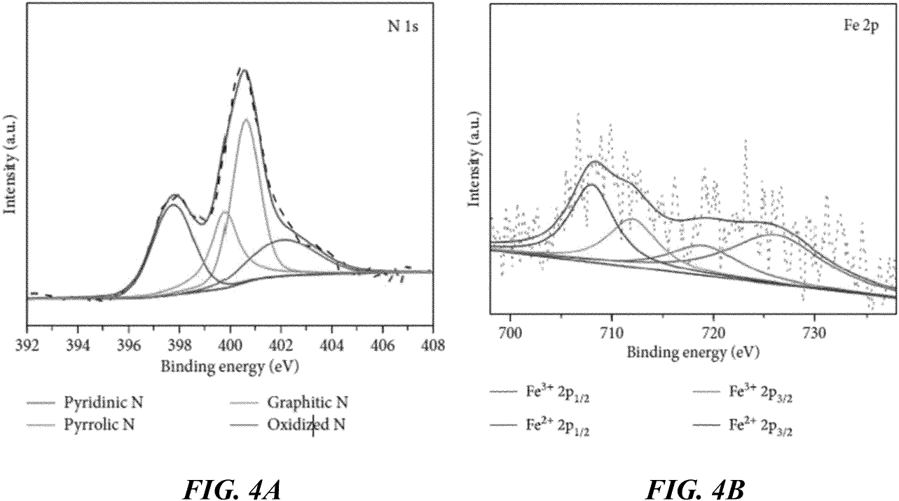

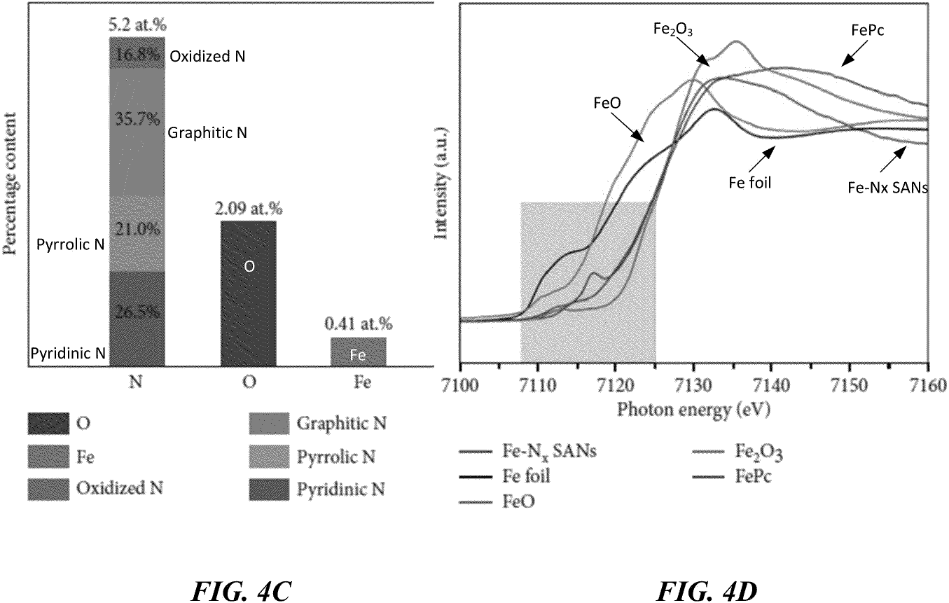

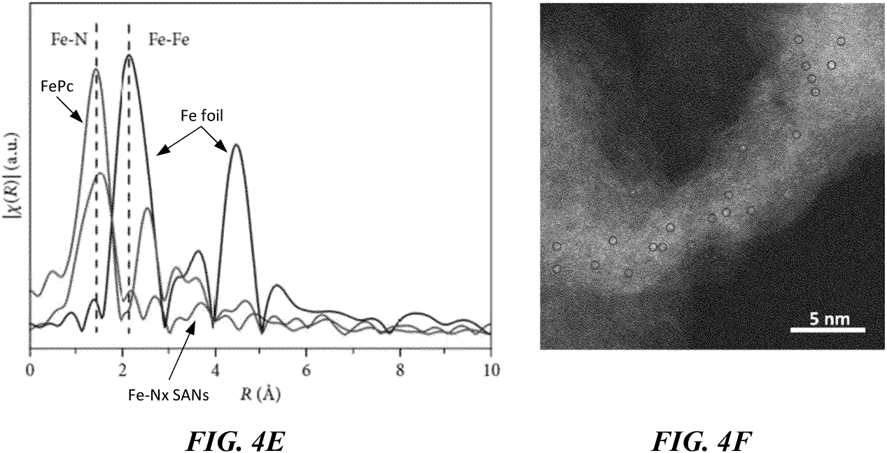

FIGS. 4A and 4B show example high-resolution nitrogen 1s and iron 2p spectra of Fe—Nx SANs, respectively, formed using the process of FIGS. 2A-2C in accordance with embodiments of the disclosed technology. As shown in FIGS. 4A and 4B, for N 1s, the spectrum of Fe—Nx SANs could be fitted into four peaks at 397.7 eV, 399.7 eV, 400.7 eV and 402.1 eV, which correspond to Fe—Nx or pyridinic N, pyrrolic N, graphitic N, and oxidized N, respectively. Here, the pyridinic N and Fe—Nx were fitted in one peak because of the small difference in binding energy between Fe—Nx and pyridinic N. For Fe 2p, four peaks of 707.9 eV, 712.1 eV, 718.9 eV, 723.5 and 725.9 eV were assigned to Fe²⁺ 2p_{⅔}, Fe³⁺2p_{⅔}, Fe²⁺2p_{½} and Fe³⁺2p_{½} based on binding energies, respectively. The deconvolution method using Gaussian-Lorentz curve fittings was adopted to conduct the semiquantitative analysis of all the elements.

FIG. 4C shows example of nitrogen, oxygen, and iron contents in Fe-Nx SANs formed using the process of FIGS. 2A-2C in accordance with embodiments of the disclosed technology. As shown in FIG. 4C, the N and Fe contents were 5.02 atom% and 0.41 atom%, respectively. The percentage of defective N configurations (e.g., pyridinic and pyrrolic N) regarded as coordination sites for single Fe atoms was high. Moreover, compared to other PPy nanotube with Fe—N—C active sites, the nanoconfinement strategy enhanced Fe loading significantly from about 0.35 atom% to about 0.41 atom%.

FIG. 4D shows an example of iron K-edge X-ray absorption near-edge structure (XANES) spectra of Fe—Nx SANs formed using the process of FIGS. 2A-2C in accordance with embodiments of the disclosed technology and reference samples of FePc, Fe foil, FeO, and Fe₂O₃. As shown in FIG. 4D, the near-edge absorption energy Fe—Nx SANs located between standard bi-(FeO) and trivalent (Fe₂O₃) iron, illustrating that +2 and +3 iron coexisted in Fe—Nx SANs, consistent with XPS results shown in FIG. 4B. Fourier-transform extended X-ray absorption fine structure (EXAFS) curve of Fe—Nx SANs in FIG. 4E showed the Fe—N peak at 1.4 Å while no Fe—Fe peak at 2.1 Å was observed. Moreover, from the K-edge EXAFS oscillations, the spectrum of Fe—Nx SANs was distinct from those of Fe foil and Fe oxides, but almost the same as that of Fe single atom reference FePc, as shown in FIG. 3K, indicating that the iron (Fe) atoms were likely atomically dispersed in Fe-Nₓ SANs. Such a structure is also like natural HRP as shown in FIG. 3J, and thus possessing peroxidase activity.

FIG. 4F shows an example scanning TEM image of Fe—Nx SAN formed using the process of FIGS. 2A-2C in accordance with embodiments of the disclosed technology. FIG. 4F showed that the Fe species were uniformly dispersed in the PPy matrix and formed single-atom active sites containing iron (Fe), which were the circled dots in FIG. 4F. In addition, no nanoparticles were observed at the atomic level, which indicated that no aggregated Fe species existed in Fe—Nx SANs. As such, FIG. 4F shows that the enriched atomic Fe—Nx moieties had been doped in the PPy matrix effectively.

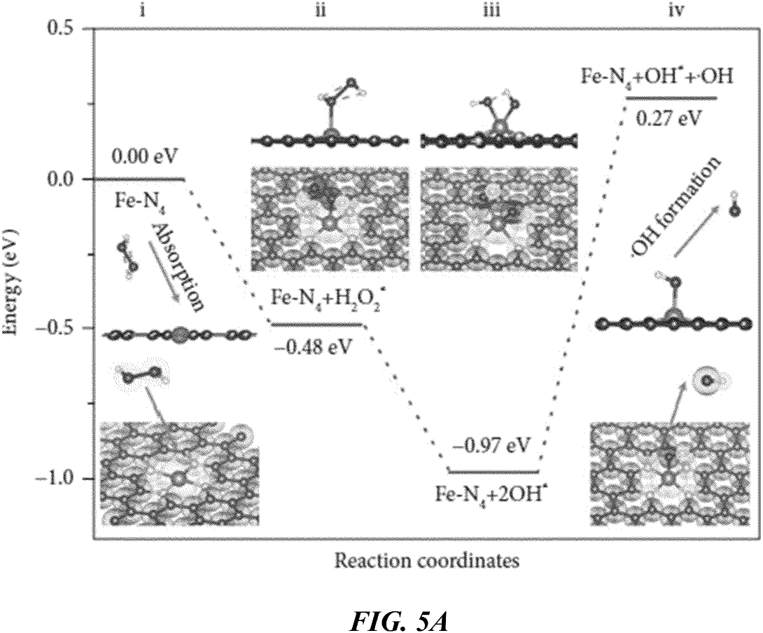

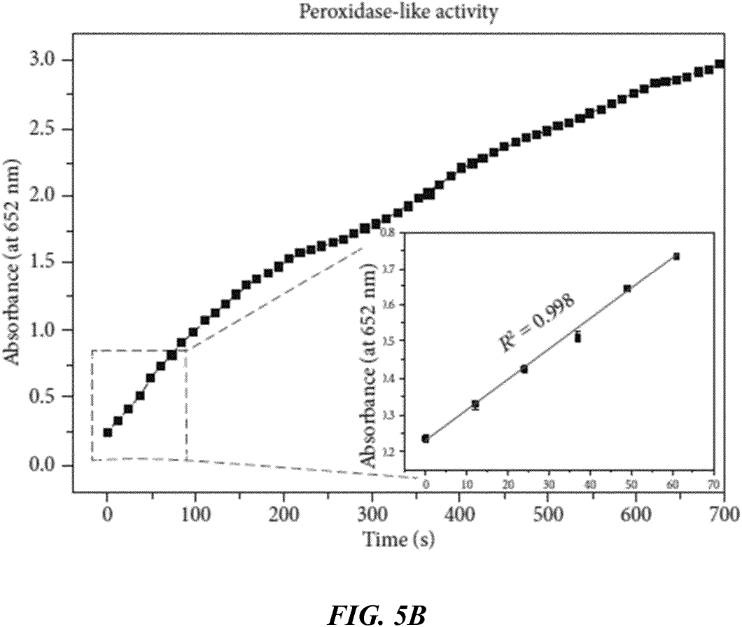

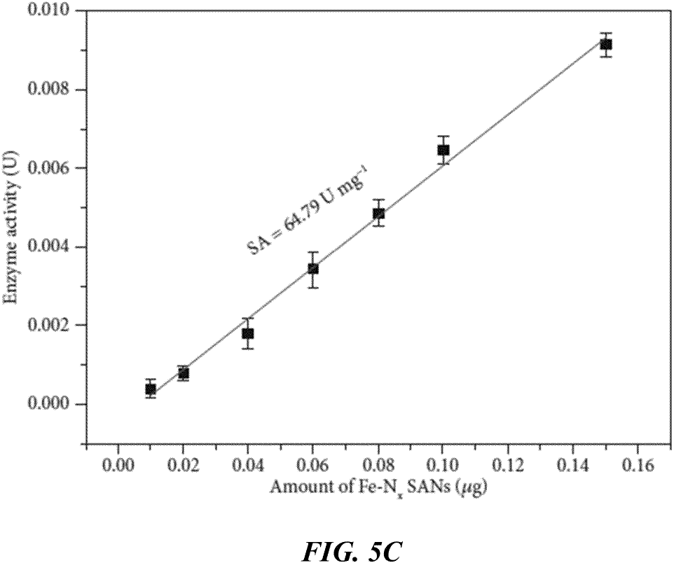

FIG. 5A is a schematic free energy diagram illustrating a catalytic operation of Fe-Nₓ SANs formed using the process of FIGS. 2A-2C in accordance with embodiments of the disclosed technology. To analyze the possible peroxidase-like catalytic property of Fe-Nₓ SANs, density functional theory calculations were performed to investigate the reaction process of the generation of hydroxyl radicals through catalyzing H₂O₂ with Fe—N₄ SANs. As shown in FIG. 5A, the H₂O₂ molecule can be initially adsorbed on a Fe active site in the Fe—N₄ SAN with an adsorption energy of -0.48 eV. Then, the H₂O₂ molecule dissociates to release a hydroxyl group from the adsorption site. Thus, an active hydroxyl radical is generated while a hydroxyl group is adsorbed at the Fe—N₄ active site. The energy diagram shows that the calculated reaction energy from the initial adsorption to the final release of hydroxyl radial was 0.27 eV. Such low reaction energy indicates peroxidase-like catalytic property of Fe—Nx SANs. Further specific peroxidase-like activities and steady-state kinetics properties of Fe—Nx SANs were assessed in acetate buffer (pH=3.6).

In further experiments, 3,3′,5,5′-tetramethylbenzidine (TMB) was employed as a substrate. First, the TMB chromogenic reaction curve of absorbance to time was obtained and the sample without adding H₂O₂ was used as a reference. The result was shown in FIG. 5B. As shown in FIG. 5B, the absorbance at 652 nm increased with reaction time and the absorbance to reaction time was generally linear in the first minute with R² coefficient close to 1 in linear regression analysis.

The catalytic activity of Fe—Nx SANs expressed in units (U) was further assessed. Specifically, different amounts Fe—Nx SANs were used to trigger chromogenic reaction of TMB. The first 60 seconds was chosen as an initial time, and the results are shown in FIG. 5C. As shown in FIG. 5C, the peroxidase-mimic activity of the Fe—Nx SANs was calculated to be 64.79 U mg⁻¹, which is higher than that of the reported Fe—Nx/SAN and conventional nanozymes. It is believed that the active sites of Fe—Nx SANs have similarly effective structures to natural enzymes. Moreover, owing to the presence of single atom Fe, the atomic utilization of Fe could be theoretically 100%. In other words, every single Fe atom can act as an active site to catalyze the oxidation reaction of hydrogen peroxide (H₂O₂). The table below shows a comparison of example peroxidase-like specific activity (U/mg) of FeNx to other published nanozymes and natural HRP:

| Enzyme | Peroxidase-like specific activity (U/mg) |
|---|---|
| Fe—Nx SANs | 64.79 |
| Fe—Nx/SAN | 57.76 |
| Fe SAEs | 6.75 |
| Fe—MOF | 5.086 |
| Go/Fe—MOF | 7.689 |
| Fe₃O₄ NPs | 5.143 |
| Carbon NPs | 3.302 |
| Au NPs | 1.633 |
| Natural HRP | 297 |

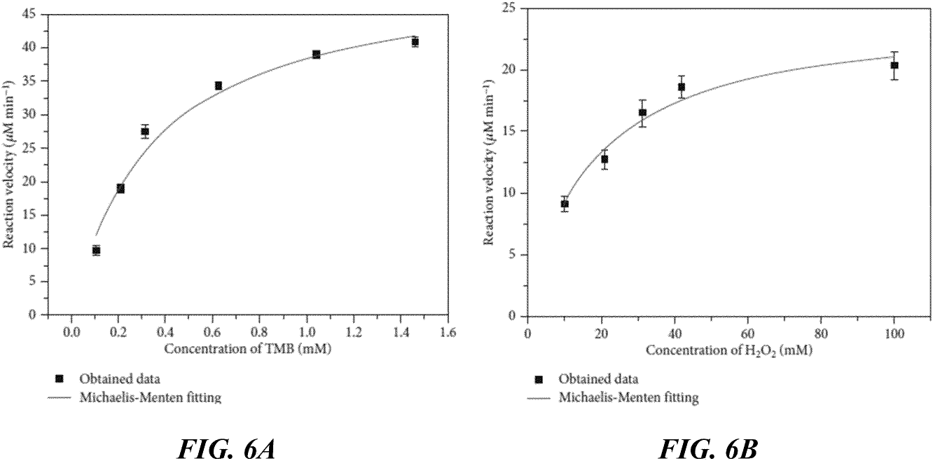

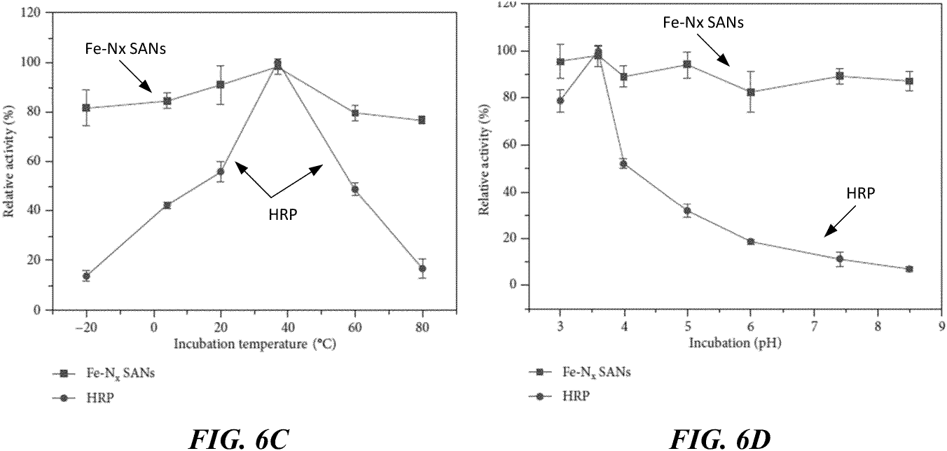

FIGS. 6A and 6B are example steady-state kinetics curves of Fe-Nₓ SANs toward TMB and H₂O₂, respectively, in accordance with embodiments of the disclosed technology. The kinetics of peroxidase-mimicking catalysis of Fe—Nx SANs were analyzed, FIGS. 6A and 6B illustrate example steady-state kinetics curves of Fe—Nx SANs towards TMB substrates and H₂O₂. Moreover, Michaelis constants (Km) of the steady-state kinetics were obtained by fitting in the Michaelis-Menten model and compared with that of HRP. The Km of Fe—Nx SANs with TMB and H₂O₂ as the substrate is slightly lower than that of HRP, demonstrating that the synthesized SANs had a comparable affinity of HRP. The table below shows a comparison of steady-state kinetics parameters of example Fe—Nx SANs and natural HRP:

| Materials | [E] (M) | Substrate | Kₘ (mM) | Vmax (µM min⁻¹) | Kcat (min⁻¹⁾ | K_{cat}/Kₘ (M⁻¹ min⁻¹) |
|---|---|---|---|---|---|---|
| | | H₂O₂ | 17.12 | 24.48 | 3.35×10⁵ | 19.57×10⁶ |
| Fe—Nx SANs | 7.3×10⁻¹¹ | | | | | |
| | | TMB | 0.3322 | 51.4 | 7.04×10⁵ | 21.19×10⁸ |
| | | H₂O₂ | 18.64 | 48.6 | 1.99×10⁶ | 10.6×10⁷ |
| Natural HRP | 2.5×10⁻¹¹ | | | | | |
| | | TMB | 0.4269 | 55.49 | 2.22×10⁶ | 5.2×10⁹ |

Also, the stability of Fe—Nx SANs in harsh environments was evaluated, shown in FIGS. 6C and 6D. As shown in FIGS. 6C and 6D, the curves appear to show that the SANs maintained excellent stability with pH and temperature variations while HRP gradually lost its activities when pH was higher than four or the temperature was lower than about 40° C. These results indicate that the example Fe—Nx SANs have much better robustness in harsh environments.

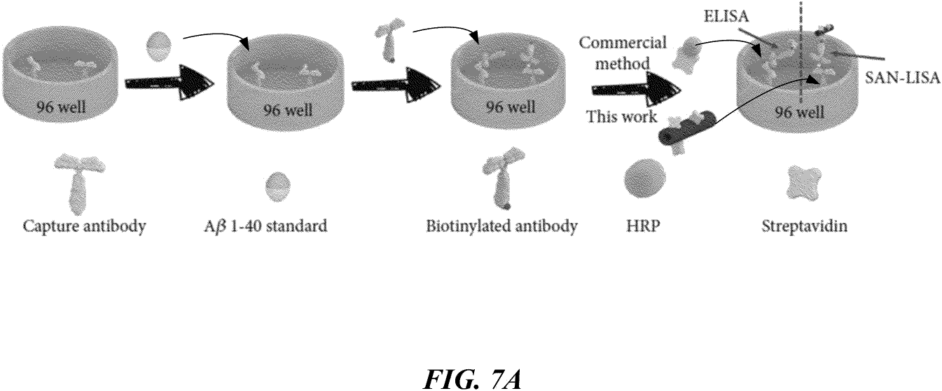

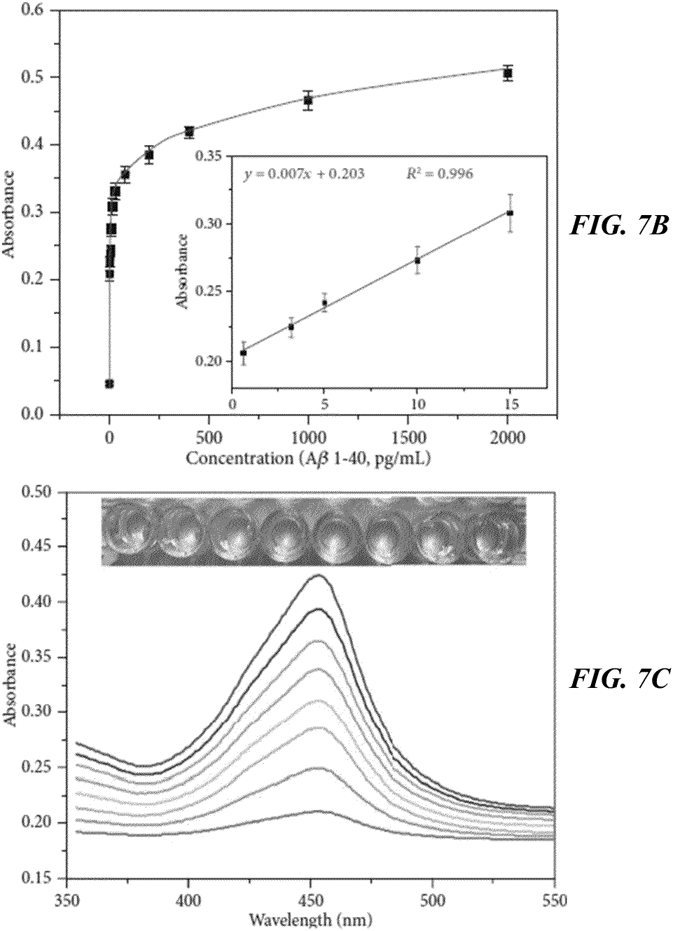

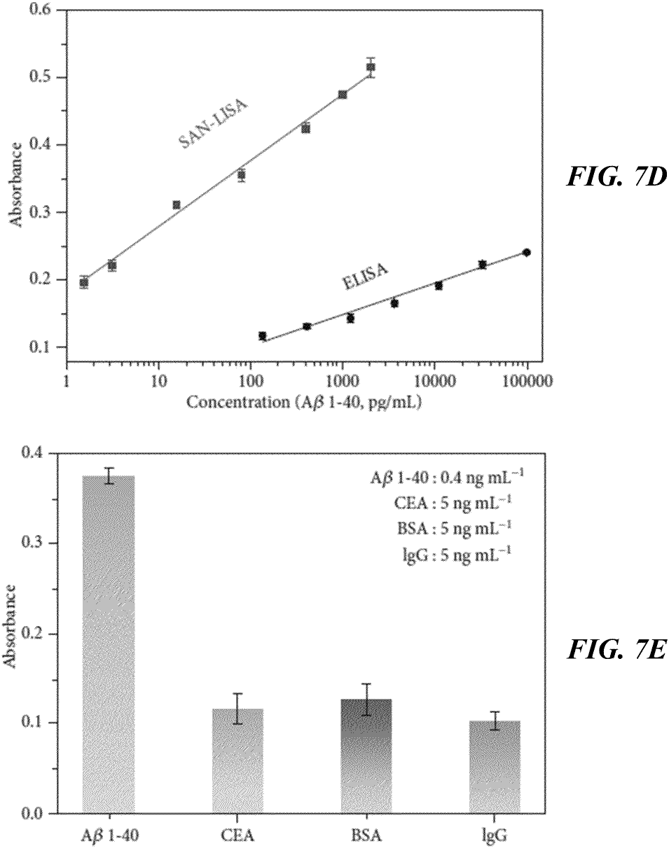

FIG. 7A is a schematic illustration of SANs-linked immunosorbent assay (SANs-LISA) for the detection of Aβ 1-40 utilizing the ELISA technique in accordance with embodiments of the disclosed technology. As shown in FIG. 7A, a capture antibody was first added to the 96-well assay. Subsequently, different amounts of Aβ 1-40 standard were added into the 96-well assay and incubated at 37° C. for 2.5 hours. As such, the Aβ 1-40 would bind with the capture antibody selectively in the wells. Each well was then washed for three times, and then 200 µL of PBST (PBS containing 0.5 wt.% of TWEEN-20) containing 1 wt.% BSA was added into the wells to block the unbonded capture antibodies at 37° C. for 1.5 hours.

Then, 100 µL of the prepared biotinylated amyloid beta 1-40 was added to each well and incubated for 1 hour with gentle shaking. Then, the wells were washed with a buffer three times. Then, 50 µL of SA labeled Fe—Nx SANs or SA labeled HRP was added into each well and shaken for 45 minutes to bind with biotin on the amyloid beta 1-40. Then, a chromogenic reaction was conducted by adding 100 µL of a combination of TMB and hydrogen peroxide (H₂O₂) to each well and the mixture was incubated for 10 minutes at room temperature under gentle shaking. Then 50 µL stop solution was added to stop the reaction. Absorbance data were collected at 450 nm immediately upon color change.

FIG. 7B shows a curve of SANs detecting Aβ 1-40 obtained during the experiment. The linear range was 1 picogram/milliliter to 2000 picogram/milliliter. The low concertation range between 0-15 picogram/milliliter is shown in inserted figure of FIG. 7B. By applying to the equation 3 S/K, where S and K referred to the standard deviation of blank sample and slope of the standard curve respectively, the limit of detection (LOD) was calculated to be 0.88 picogram/milliliter. Absorbance spectra of various concentrations of Aβ 1-40 detected by SANs-LISA and their corresponding colorimetric signal were shown in FIG. 7C. As shown clearly in FIG. 7C, the signal intensities increased with elevated concentrations of Aβ 1-40. As a comparison, the calibration curve of commercial ELISA for the Aβ 1-40 detection was analyzed. By applying the previous equation, the LOD of the traditional ELISA was calculated to be 9.98 picogram/milliliter, which was almost ten times higher than that utilizing SANs-LISA.

It is believed that the enhanced sensitivity was due to the ultrahigh surface area of the nanotubes 106 (FIG. 1) that can host large numbers of active sites. Furthermore, the sensitivity of SANs-LISA was evaluated by comparing the signal between traditional ELISA and proposed SANs-LISA. As shown in FIG. 7D, SANs-LISA appeared to have better sensitivity with much higher absorbance on much lower concertation of Aβ 1-40. Moreover, the detection performance of the two methods with the same Aβ 1-40 concentrations were studied. As shown in the table below, compared with previously reported detection results of Aβ 1-40 using different methods, the proposed SANs-LISA method exhibits superior detection performance.

| | | |
|---|---|---|
| Techniques | LOD (pg/ml) | Linear Range (pg/ml) |
| SANs-LISA | 0.88 | 1-2000 |
| Electrochemical Immunoassay | 19 | 20-12500 |
| Square Wave Voltammetry at Glassy Carbon Electrode | 7×10⁵ | nonlinear |
| Microfluidic Droplet | 2165 | NP |
| Electrochemical Impedance Spectroscopy | 2468 | 43.3-4.33×10⁵ |
| Surface Plasmon Resonance | 86.6 | 86.6-865.9 |
| Square Wave Voltammetry | 8.6×10⁵ | 1.772×10⁶-8.66×10⁶ |

Lastly, the specificity of SANs-LISA was analyzed, as displayed in FIG. 7E. As shown in FIG. 7E, Aβ 1-40 exhibited a distinct signal, while the other competition protein had negligible signals, indicating the satisfactory specificity of SANs-LISA.

From the foregoing, it will be appreciated that specific embodiments of the disclosure have been described herein for purposes of illustration, but that various modifications may be made without deviating from the disclosure. In addition, many of the elements of one embodiment may be combined with other embodiments in addition to or in lieu of the elements of the other embodiments. Accordingly, the technology is not limited except as by the appended claims.

I/We claim:

1. A method of making a single atom nanozyme linked immunoassay, the method comprising:
forming a soft template having multiple nanoscale structures in an aqueous solution;
adding a solution of a monomer and a solution of a metal containing salt into the aqueous solution such that the metal containing salt causes polymerization of the monomer to form multiple nanostructures according to the nanoscale structures of the previously formed soft template in the aqueous solution;
upon forming the multiple nanostructures, coating the individual nanostructures with a confinement layer in the aqueous solution, the confinement layer covering at least a part of an external surface of the individual nanostructures; and
after coating the individual nanostructures with the confinement layer, pyrolyzing the nanostructures coated with the confinement layer to derive the single atom nanozyme, wherein during the pyrolyzing of the nanostructures, the confinement layer at least restricts or completely prevents migration of atoms on the external surface of the individual nanostructures.

2. The method of claim 1 wherein:
the multiple nanostructures individually having multiple active sites for catalyzing an oxidation reaction;
the confinement layer coats at least some of the multiple active sites on the individual nanostructures; and
the method further includes removing the confinement layer from the individual nanostructures after pyrolyzing the nanostructures.

3. The method of claim 1 wherein:
the multiple nanostructures individually have multiple active sites for catalyzing an oxidation reaction, the individual active sites having a single atom of the metal in the metal containing salt covalently connected to additional atoms of the polymerized monomer;
the confinement layer coats at least some of the multiple active sites having the single atom of the metal in the metal containing salt; and
the method further includes removing the confinement layer from the individual nanostructures after pyrolyzing the nanostructures.

4. The method of claim 1 wherein the confinement layer includes a layer of magnesium oxide (MnO₂), silicon oxide (SiO₂), or titanium oxide (TiO₂) on the individual multiple nanotubes.

5. The method of claim 1 wherein coating the individual nanostructures includes:
adding potassium permanganate (KMnO₄) to the aqueous solution upon forming the multiple nanostructures; and
reducing the added potassium permanganate (KMnO₄) to form a magnesium oxide (MnO₂) coating on the external surface of the individual nanostructures.

6. The method of claim 1 wherein:
the nanostructures include multiple nanotubes;
adding the solution of the monomer and the solution of the metal containing salt includes adding a solution of pyrrole monomer and a solution of iron chloride (FeCl₃) to the aqueous solution such that the iron chloride (FeCl₃) causes polymerization of the pyrrole monomer to form the multiple nanotubes of polypyrrole according to the nanoscale structures of the previously formed soft template in the aqueous solution; and
coating the individual formed nanotubes includes:
adding potassium permanganate (KMnO₄) to the aqueous solution upon forming the multiple polypyrrole nanotubes; and
reducing the added potassium permanganate (KMnO₄) to form a magnesium oxide (MnO₂) coating on the external surface of the individual formed polypyrrole nanotubes.

7. The method of claim 1 wherein:
the nanostructures include multiple nanotubes;
adding the solution of the monomer and the solution of the metal containing salt includes adding a solution of pyrrole monomer and a solution of iron chloride (FeCl₃) to the aqueous solution such that the iron chloride (FeCl₃) causes polymerization of the pyrrole monomer to form the multiple nanotubes of polypyrrole according to the nanoscale structures of the previously formed soft template in the aqueous solution, wherein the individual polypyrrole nanotubes having multiple active sites each having a single iron (Fe) atom covalently connected to additional nitrogen (N) atoms which in turn are covalently connected to additional carbon (C) atoms of the polypyrrole; and
coating the individual formed nanotubes includes:
adding potassium permanganate (KMnO₄) to the aqueous solution upon forming the multiple polypyrrole nanotubes; and
reducing the added potassium permanganate (KMnO₄) to form a magnesium oxide (MnO₂) coating on at least some of the active sites at the external surface of the individual formed polypyrrole nanotubes.

8. The method of claim 1 wherein:
the nanostructures include multiple polypyrrole nanotubes;
adding the solution of the monomer and the solution of the metal containing salt includes adding a solution of pyrrole monomer and a solution of iron chloride (FeCl₃) to the aqueous solution such that the iron chloride (FeCl₃) causes polymerization of the pyrrole monomer to form multiple nanotubes of polypyrrole according to the nanoscale structures of the previously formed soft template in the aqueous solution, wherein the individual polypyrrole nanotubes having multiple active sites each having a single iron (Fe) atom covalently connected to additional nitrogen (N) atoms which in turn are covalently connected to additional carbon (C) atoms of the polypyrrole;
coating the individual formed nanotubes includes:
adding potassium permanganate (KMnO₄) to the aqueous solution upon forming the multiple polypyrrole nanotubes; and
reducing the added potassium permanganate (KMnO₄) to form a magnesium oxide (MnO₂) coating on at least some of the active sites at the external surface of the individual formed polypyrrole nanotubes; and
during the pyrolyzing of the formed nanotubes, the magnesium oxide (MnO₂) coating at least restricts or completely prevents migration of iron (Fe) atoms on the external surface of the individual formed nanotubes, thereby reducing aggregation of the iron (Fe) atoms during pyrolysis.

9. The method of claim 1, further comprising covalently linking the formed single atom nanozyme to an antibody.

10. A single atom nanozyme linked immunoassay, comprising:
an antibody configured to detect an antigen on a target analyte; and
a single atom nanozyme chemically linked to the antibody, the single atom nanozyme having a nanotube formed from a polymer, the nanotube having multiple active sites on an external surface of the nanotube for catalyzing an oxidation reaction of hydrogen peroxide (H₂O₂), wherein:
the multiple active sites individually include a single metal atom covalently connected to additional atoms of the polymer; and
an atomic concentration of the metal atoms individually incorporated into themultiple active sites is about 0.4% to about 1.0% on the external surface of the nanotube.

11. The single atom nanozyme linked immunoassay of claim 10 wherein:
the metal atom includes an iron (Fe) atom; and
the individual active sites each include a single iron (Fe) atom covalently connected to the additional atoms of the polymer.

12. The single atom nanozyme linked immunoassay of claim 10 wherein:
the metal atom includes an iron (Fe) atom;
the polymer includes polypyrrole; and
the individual active sites each include a single iron (Fe) atom covalently connected to multiple nitrogen (N) atoms, which are individually covalently connected to multiple carbon (C) atoms of the polypyrrole.

13. The single atom nanozyme linked immunoassay of claim 10 wherein the single atom nanozyme is covalently connected to the antibody or connected to the antibody via one or more intermediate proteins.

14. The single atom nanozyme linked immunoassay of claim 10 wherein the single atom nanozyme is covalently connected to streptavidin that is to bind biotinylated amyloid beta 1-40.

15. A method of detecting a biomarker using a single atom nanozyme linked immunoassay, the method comprising:
binding an antibody of the biomarker with an antibody linked to the single atom nanozyme in a sample solution, wherein the single atom nanozyme includes:
a nanotube formed from a polymer, the nanotube having multiple active sites on an external surface of the nanotube for catalyzing an oxidation reaction of hydrogen peroxide (H₂O₂), wherein:
the multiple active sites individually include a single metal atom covalently connected to additional atoms of the polymer; and
an atomic concentration of the metal atoms individually incorporated into the multiple active sites is about 0.4% to about 1.0% on the external surface of the nanotube; and
adding a substrate to the sample solution, thereby causing a change of a color of the sample solution; and
measuring the change of the color of the sample solution as corresponding to a concentration of the biomarker in the sample solution.

16. The method of claim 15 wherein:
the metal atom includes an iron (Fe) atom; and
the individual active sites each include a single iron (Fe) atom covalently connected to the additional atoms of the polymer.

17. The method of claim 15 wherein:
the metal atom includes an iron (Fe) atom;
the polymer includes polypyrrole; and
the individual active sites each include a single iron (Fe) atom covalently connected to multiple nitrogen (N) atoms, which are individually covalently connected to multiple carbon (C) atoms of the polypyrrole.

18. The method of claim 15 wherein the single atom nanozyme is covalently connected to the antibody or connected to the antibody via one or more intermediate proteins.

19. The method of claim 15 wherein the single atom nanozyme is covalently connected to streptavidin, and the method further includes detecting amyloid beta 1-40 via binding biotinylated amyloid beta 1-40.

20. The method of claim 15 wherein:
adding the substrate to the sample solution includes adding a solution containing a combination of 3,3′,5,5′-tetramethylbenzidine (TMB) and hydrogen peroxide (H₂O₂); and
the method further includes catalyzing an oxidation reaction between the 3,3′,5,5′-tetramethylbenzidine (TMB) and the hydrogen peroxide (H₂O₂) with the multiple active sites on the external surface of the nanotube, thereby causing the change in the color in the sample solution.
